(19) United States
(12) Patent Application Publication
(10) Pub. No.: US 2024/0293445 A1
(43) Pub. Date: Sep. 5, 2024

(54) SYSTEM FOR TRANSPORTING AND DELIVERING OF NITRIC OXIDE

(71) Applicant: YES2NO GMBH, Düsseldorf (DE)

(72) Inventor: Tienush Rassaf, Düsseldorf (DE)

(21) Appl. No.: 18/573,514

(22) PCT Filed: Jul. 4, 2022
(86) PCT No.: PCT/EP2022/068432

§ 371 Date: Dec. 22, 2023

(30) Foreign Application Priority Data
Jul. 2, 2021 (EP) 21183347.0

(51) Int. Cl. A61K 33/00; A61K 9/00; A61K 9/06; A61K 47/12

(52) U.S. Cl. CPC A61K 33/00; A61K 9/0014; A61K 9/06; A61K 47/12

(57) ABSTRACT

The present invention provides systems for transporting and/or delivering nitric oxide into and/or through an epithelial layer, such as skin and/or mucosa, comprising an alpha-hydroxy acid (AHA) and a nitric oxide source from which nitric oxide (NO) is generated or released.

### FIELD OF THE INVENTION

The present invention generally concerns topical applications for medical purposes. The present invention provides a delivery system for nitric oxide in applications to the body surface. In particular, an alpha-hydroxy acid is combined with a nitric oxide source, wherein the alpha-hydroxy acid effects transport of the nitric oxide into and through an epithelial layer, such as skin and mucosa.

### BACKGROUND OF THE INVENTION

Nitric oxide (NO) is a potent vasodilator synthesized and released by vascular endothelial cells, playing an important role in regulating vascular local resistance and blood flow (Palmer R M, Ferrige A G, Moncada S, Nitric oxide release accounts for the biological activity of endothelium-derived relaxing factor. Nature, 1987; 327(6122): 524-6). In mammalian cells, NO is produced along with L-citrulline by the enzymatic oxidation of L-arginine. NO is also involved in the inhibition of both platelet and leukocyte aggregation and adhesion, the inhibition of cell proliferation, the scavenging of superoxide radicals and the modulation of endothelial layer permeability. Furthermore, NO also has been shown to possess antimicrobial properties and immunomodulatory effects that may have application for management of skin disorders, including cutaneous infections, (auto-)inflammatory disorders, and wound healing. The advantageous effects of NO on wound repair may be addressed to its functional influences on angiogenesis, inflammation, cell proliferation, matrix deposition, and remodeling (Minniti C P, Gorbach A M, Xu D, Hon Y Y, Delaney K M, Seidel M, et al., Topical sodium nitrite for chronic leg ulcers in patients with sickle cell anaemia: a phase 1 dose-finding safety and tolerability trial. Lancet Haematol, 2014; 1(3): e95-e103; Fang F C, Perspectives series: host/pathogen interactions. Mechanisms of nitric oxide-related antimicrobial activity. J Clin Invest, 1997; 99(12): 2818-25). Many dermatologic diseases have been associated to changes in NO activity or may involve inflammation pathways that could be therapeutically regulated by NO. Therefore, the development of a topical NO formulation could make a huge impact in the field of dermatology (del Rosso J, Kircik L, Spotlight on the Use of Nitric Oxide in Dermatology: What Is it? What Does it Do? Can It Become an Important Addition to the Therapeutic Armamentarium for Skin Disease? J Drugs Dermatol, 2017; 16(1): s4-10).

Various systems for the generation of NO in topical formulations have been described in the art, for example nitrate/nitrite/nitroso compounds which release NO on exposure to changes in pH, certain endogenous enzymes, diazeniumdiolates, S-nitrosothiols, and vehicles combined with NO-donors such as nanoparticles, polymers, dendrimers, micelles or lipid-based nanocarriers (Liang H, Nacharaju P, Friedman A, Friedman J M, Nitric oxide generating/releasing materials. Future Sci OA, 2015; 1(1); Bryan N S, Nitric oxide enhancement strategies. Future Sci OA, 2015; 1(1): FSO48).

Most of these systems did not succeed because they possess various disadvantages including formation of harmful byproducts, inducing allergic reactions or skin irritations, inconvenient application methods, an expensive production process (Adler B L, Friedman A J, Nitric oxide therapy for dermatologic disease. Future Sci OA, 2015; 1(1): FSO37) and/or unsatisfactory NO delivery (Oplander C, Romer A, Paunel-Gorgulu A, Fritsch T, van Faassen E E, Murtz M, et al., Dermal application of nitric oxide in vivo: kinetics, biological responses, and therapeutic potential in humans. Clin Pharmacol Ther, 2012; 91(6): 1074-82).

Most of the disadvantages mentioned above vary from substance to substance and might be solved by fine-tuning the material, but the unsatisfactory NO delivery is a major problem for most of the different NO-generating materials especially in topical applications. For clinical interest, a potential drug delivery system that provides the necessary stability and release characteristics of NO donors to make it an effective and safe topical therapeutic agent which is cost-effective and easy to use is a main desideratum.

Therefore, the present invention comprises a unique transport delivery system which could be utilized and implemented in any NO-generating material to enhance NO delivery to the target site.

### SUMMARY OF THE INVENTION

As explained above, nitric oxide (NO) is an important mediator in many physiological and pathophysiological mechanisms in e.g., the cardiovascular system, the immune system, the neuronal system, and the skin, respectively. In the latter case, a topical application of NO may have anti-inflammatory, immunomodulatory, antimicrobial, and vasoactive properties.

The epidermis of the skin, however, presents a natural barrier, which inhibits the penetration of NO into the deeper skin layers (dermis, hypodermis), especially in higher amounts. This requires the application of very high pharmacological topical NO concentrations to enable even small amounts of NO to enter the skin.

To overcome the drawbacks of the prior art, a novel and unique transport delivery system for nitric oxide (NO) for topical applications is presented. It ensures skin penetration and delivery of NO into the deeper skin layers and can be added to any NO-generating and NO-releasing substances (e.g., authentic NO-solution, organic nitrates (mono-, di-, trinitrate), acidified nitrite, etc.), respectively, to enhance the amount of NO reaching the target site.

According to a first aspect, the present invention provides a system for transporting and/or delivering nitric oxide into and/or through an epithelial layer, such as skin and/or mucosa, comprising an alpha-hydroxy acid and a nitric oxide source.

The system according to the first aspect of the invention comprises the use of alpha-hydroxy acids (AHA) as a transport delivery system for NO. The AHA catches the NO generated in situ and transports it into deeper skin layers (see FIG. 1).

According to a second aspect, the present invention provides the system according to the first aspect for use in medicine.

According to a third aspect, the present invention provides the system according to the first aspect for use in the treatment of infections, inflammations, injuries, skin disorders and/or blood vessel disorders, such as chronic inflammatory skin diseases, acne, eczema, neurodermatitis, psoriasis, scars, wrinkles, abrasions, burns, trauma, hematoma, androgenetic alopecia (male-pattern hair loss and female-pattern hair loss), blepharitis, Raynaud syndrome, bacterial skin & soft-tissue infections, leg ulcer causes by peripheral vascular disease, chronic venous insufficiency and/or ischemic vasculitis.

Alpha-hydroxy acids, such as glycolic acid, increase skin hydration and skin turgor as well as normalize epidermal differentiation. They interfere with the intercellular ionic bonding in the stratum corneum of the epidermis, thus inducing keratolysis and a reduction in thickness and diffusion through the skin has been shown in multiple studies (Tang S C, Yang J H, Dual Effects of Alpha-Hydroxy Acids on the Skin. Molecules, 2018; 23(4); Babilas P, Knie U, Abels C, Cosmetic and dermatologic use of alpha hydroxy acids. J Dtsch Dermatol Ges, 2012; 10(7): 488-91; Jiang M, Qureshi S A, Assessment of in vitro percutaneous absorption of glycolic acid through human skin sections using a flow-through diffusion cell system. Journal of Dermatological Science, 1998; 18(3): 181-188; Kraeling M E K, Bronaugh R L, In vitro percutaneous absorption of alpha hydroxy acids in human skin. J Soc Cosmet Chem, 1997; 48: 187-197; Van Scott E J, Yu R J, Hyperkeratinization, corneocyte cohesion, and alpha hydroxy acids. Journal of the American Academy of Dermatology, 1984; 11(5): 867-879). The system according to the present invention can be used in all kinds of preparations to increase the amount of NO reaching the target site, enhancing the effectivity and counteracting the poor NO delivery in other NO-generating formulations.

Alpha-hydroxy acids-especially in low nanomolar up to molar concentrations-which can be used for this purpose include, but are not limited to, lactic acid, citric acid, citric acid monohydrate, mandelic acid, glycolic acid, tartaric acid, and malic acid. The present inventors have found that NO transportation significantly increases when alpha-hydroxy acids are added to NO topical applications.

The present disclosure refers to the addition of the transport delivery system to formulations, which can be applied to the skin and can be used for dermatological or cosmetic purposes but is not limited by the application method. The formulations with the addition of the alpha-hydroxy acid can be applied particularly (but not exclusively) to every kind of skin dressing including patches, plasters, bandings, pads, compresses, folios, etc., for the use on compromised (e.g., by trauma or disease) and non-compromised (e.g. wrinkled) skin. It can further be applied without skin dressing as gel or aqueous solution. Depending on the application method, the wide application possibilities include a range in liquidity from jellylike to liquid aqueous solutions.

According to a fourth aspect, a dressing is provided comprising the system according to the first aspect.

According to a fifth aspect, the present invention provides a method for enhancing the transport and/or delivery of nitric oxide into and/or through an epithelial layer, such as skin and/or mucosa, the method comprising the steps of:
(i) mixing a nitric oxide source with an alpha-hydroxy acid,
(ii) applying the nitric acid source to the epithelial layer,
(iii) activating the nitric acid source to generate and/or release nitric oxide.

According to a sixth aspect, the present invention provides the use of an alpha-hydroxy acid for enhancing the transport and/or delivery of nitric oxide into and/or through an epithelial layer, such as skin and/or mucosa. Other objects, features, advantages and aspects of the present application will become apparent to those skilled in the art from the following description and appended claims.

### BRIEF DESCRIPTION OF THE FIGURES

FIG. 1: Graphical presentation of the penetration of NO or lack thereof through the epidermis of the skin. Without the transporter delivery system described in this invention, the NO molecules do not reach deeper skin layers (dermis, hypodermis) since the epidermis provides a natural barrier. The transporter (alpha-hydroxy acid) carries the NO generated through the epidermis of the skin to the target site (adapted from Servier Medical Art by Servier (licensed under a Creative Commons Attribution 3.0 Unported License)).

FIG. 2: Graph of time (in seconds) versus concentration of NO (in parts per billion, ppb) showing the amount of NO generated by mixing the Gel A and Gel B.

FIG. 3: (A) The skin of a patient before (left) and after (right) eight weeks of application of the NO-generating gel with the transport delivery system. (B) Change in Global Acne Grading System (GAGS) score in said patient group after four and eight weeks.

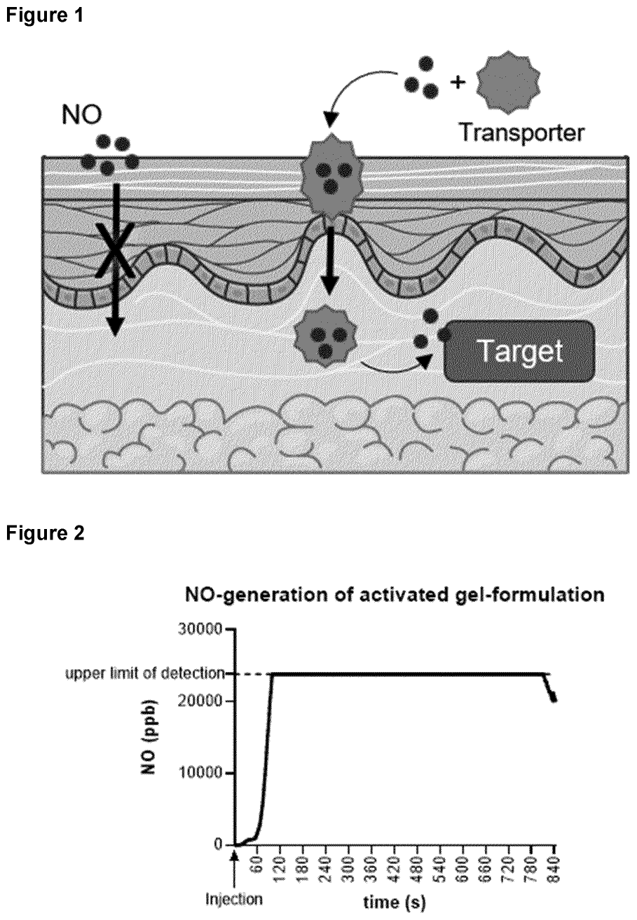

### DETAILED DESCRIPTION OF THE INVENTION

The different aspects and embodiments of the invention disclosed herein make important contributions to the art as is also explained in the following.

### The System According to the First Aspect

According to the first aspect, a system is provided for transporting and/or delivering of nitric oxide into and/or through an epithelial layer, such as skin and/or mucosa, comprising an alpha-hydroxy acid and a nitric oxide source.

As described above, alpha-hydroxy acids increase epidermal penetration. According to the first aspect, alpha-hydroxy acid is used to transport and deliver nitric oxide through epithelial layers that inhibit or impair nitric oxide penetration into deeper tissue layers. In particular, nitric oxide is generated and/or released from the nitric oxide source. The generation and/or release of nitric oxide from the nitric oxide source may be induced by an acidic pH. The pH may be adjusted by the addition of an acid to the nitric oxide source, such as the alpha-hydroxa acid or other acid, e.g. sulfuric acid. Other nitric oxide sources where generation and/or release of NO is induced by other mechanisms are also encompassed.

According to one embodiment, the system may be a one-component system comprising the alpha-hydroxy acid and the nitric oxide source in one composition. According to this embodiment, the system may comprise further additives and/or a suitable nitric oxide source to prevent nitric oxide generation and/or release until the system is applied to the epithelial layer.

According to an alternative embodiment, the system is a two-component system which comprises a first composition comprising the alpha-hydroxy acid and a second composition comprising the nitric oxide source. According to this embodiment, upon contact of the first composition and the second composition nitric oxide is generated and/or released.

As used herein, the term “epithelial layer” in particular refers to any surface of the body, especially of the human body, including outer surfaces such as the skin, and inner surfaces such as mucosal membranes. The epithelial layer is generally characterized by a reduced permeability for nitric oxide. According to one embodiment, the epithelial layer is the epidermis of the skin. The nitric oxide is transported and/or delivered by the alpha-hydroxy acid into the dermis and/or hypodermis, i.e. the deeper skin layers. As explained in the summary of the invention, the epidermis presents a natural barrier, which inhibits penetration of nitric oxide into the deeper skin layers, such as the dermis and hypodermis. Accordingly, the system according to the first aspect can be advantageously used to transport and/or deliver nitric oxide through the epidermis and in particular into the dermis and/or hypodermis. According to an alternative embodiment, the epithelial layer is a mucosa. The mucosa may be provided by any mucous membrane, e.g. oral mucosa, nasal mucosa, intestinal mucosa, or mucous membrane of the internal organs. According to one embodiment, the system enables the transport and delivery of nitric oxide by alpha-hydroxy acid into and/or through diverse epithelial layers, including skin and mucosa as described above.

The individual properties and preferred embodiments of the system according to the first aspect will now be described in detail.

### The Alpha-Hydroxy Acid

The system according to the first aspect comprises an alpha-hydroxy acid. According to one embodiment, the system may comprise one or more alpha-hydroxy acids. Alpha-hydroxy acids comprise a hydroxyl group on the carbon atom adjacent to the carboxyl group and may be natural occurring, such as in fruits, sugar cane and milk, or may be synthetic.

According to one embodiment, the alpha-hydroxy acid is of the general formula:

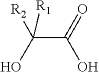

In particular, R1 and R2 are independently selected from the group consisting of hydrogen, alkyl, alkenyl, aryl, hydroxy, carboxy and carbonyl, wherein alkyl, alkenyl and aryl may optionally be substituted with one or more of alkyl, alkenyl, aryl, hydroxy, carboxy and carbonyl.

Alkyl especially refers to C1 to C5 alkyl, in particular C1 to C3 alkyl. Alkenyl especially refers to C2 to C5 alkenyl, in particular C2 to C3 alkenyl. Aryl especially refers to phenyl.

According to one embodiment R₁ is H and R₂ has one of the following characteristics:
(i) R₂ is H (glycolic acid);
(ii) R₂ is CH₃ (lactic acid);
(iii) R₂ is C₂H₃O₂ (malic acid);
(iv) R₂ is C₆H₅ (mandelic acid);
(v) R₂ is C₂H₃O₃ (tartaric acid); or
(vi) R₂ is C₄H₅O₄ (isocitric acid).

According to one embodiment R₁ and R₂ are both C₂H₃O₂ (citric acid).

According to one embodiment, the alpha-hydroxy acid is provided by one isomer, such as stereoisomer or enantiomer. According to one embodiment, the alpha-hydroxy acid is provided by a combination of isomers.

According to one embodiment, the alpha-hydroxy acid is selected from the group consisting of—but are not limited to—glycolic acid, lactic acid, citric acid, isocitric acid, citric acid monohydrate, mandelic acid, tartaric acid, malic acid, tartronic acid, hydroxycaprylic acid, hydroxycapric acid and mixtures thereof. Glycolic acid has the smallest molecular size of alpha-hydroxy acids and is known to have the greatest bioavailability and skin penetration. According to an advantageous embodiment, the alpha-hydroxy acid is glycolic acid. As is demonstrated by the examples, glycolic acid is a suitable alpha-hydroxy acid to transport and deliver nitric oxide through the epidermis of the skin to deeper skin layers. The skilled person can choose suitable alpha-hydroxy acids for the system according to the first aspect.

### The Nitric Oxide Source

The system further comprises a nitric oxide source from which nitric oxide can be generated and/or released. Such a nitric oxide source may be provided by salts (M⁺NO₂⁻, wherein M is a monovalent cation) and esters (R—O—N═O, wherein R is an organic radical) of nitrous acid HNO₂. Examples of salts are potassium nitrite (KNO₂), sodium nitrite (NaNO₂) or calcium nitrite (Ca(NO₂)₂). Suitable nitric oxide sources for clinical topical formulations showing a good biocompatibility are well-known in the prior art and the skilled person can choose a suitable nitric oxide source for the system according to the first aspect of the present invention.

According to one embodiment, the nitric oxide source is selected from the group consisting of authentic nitric oxide, nitric oxide solution, organic and inorganic nitrates, such as mono-nitrate, di-nitrate and tri-nitrate, acidified nitrite, nitrogen oxides, and mixtures thereof. “Acidified nitrite” refers to any nitrite that reacts with an acid, including alpha-hydroxy acid, to instable nitrous acid (HNO₂) which decomposes inter alia into nitric oxide (NO).

NO is generated and/or released from the nitric oxide source. According to one embodiment, this may be achieved by an acidic pH. For instance, the pH of the first composition of the two-component system may be in the acidic range, e.g. due to the alpha-hydroxy acid, such that in case the first and second compositions are combined, nitric oxide is released from the nitric oxide source of the second composition. The pH of the first composition may be adjusted according to the ratio of the first composition and the second composition which are to be mixed to ensure a pH in the acidic range after mixing which leads to the generation and release of nitric oxide from the nitric oxide source. As is demonstrated by the examples, an acidic pH leads to the generation and release of nitric oxide from suitable nitric oxide sources.

According to one embodiment, the nitric oxide source is an acidified inorganic nitrite. According to a preferred embodiment, the nitric oxide source is sodium nitrite (NaNO₂). As demonstrated by the examples, sodium nitrite is a suitable nitric oxide source for the generation of nitric oxide at physiological conditions.

### Further Components of the System

According to one embodiment, the system further comprises an activator substance which initiates release of nitric oxide from the nitric oxide source. Such an activator substance may be an acid that provides an acidic pH and thus generates and releases nitric oxide from the nitric oxide source. According to one embodiment, the activator substance may be provided by the alpha-hydroxy acid of the system. According to one embodiment, the activator substance may be a different acid than alpha-hydroxy acid. According to a further embodiment, two or more acids may be comprised in the system as activator substance, optionally wherein one of the two or more acids is alpha-hydroxy acid. As is demonstrated in the examples, two or more acids, such as ascorbic acid and alpha-hydroxy acid, may provide an activator substance that initiates the release of the nitric oxide source of the system. Any activator substance can be used to initiate the release of nitric oxide from the nitric oxide source of the system according to the first aspect.

According to one embodiment, the system further comprises a solvent. Suitable solvents are known in the art, such as nonpolar, polar aprotic and polar protic solvents, e.g. water, alcohol, polyol or multicomponent solvents. The solvent may also be provided by a mixture of two or more solvents. The skilled person may choose a suitable solvent showing a good biocompatibility and low toxicity depending on the desired formulation, including gels, creams, lotions, foams, or sprays. According to an advantageous embodiment, the solvent is water.

According to one embodiment, the system further comprises at least one thickening agent. According to one embodiment, the thickening agent is a gelling agent. According to this embodiment, the solvent and the thickening agent provide the preparation of the system according to the first aspect for different formulations, including gels, creams, lotions, foams, and sprays. Thus, the system can be prepared for every type of formulation, which can be applied to an epithelial layer, such as skin and/or mucosa. The thickening agent may be natural or synthetic. Various biocompatible thickening agents, including gelling agents, suitable for topical application are known in the art, e.g. polyacrylic acid (PAA), polyethylene glycol, cellulose-derived thickening agents (such as hydroxyethylcellulose), gelatin, xanthan gum, and hyaluronic acid. Thus, the skilled person may choose any biocompatible and non-toxic thickening agent and/or combination of solvent and thickening agent depending on the desired formulation.

According to one embodiment, the system comprises PAA.

According to one embodiment, the system comprises hyaluronic acid. According to an advantageous embodiment, the hyaluronic acid is provided by hyaluronic acids of different molecular weights.

According to one embodiment, the system may comprise one or more further skin penetration enhancers. Suitable skin penetration enhancers are well described, such as pyrrolidones, alcohols (e.g., ethanol), alkanols (e.g., decanol), sulfoxides (e.g., dimethyl sulfoxide/DMSO), glycols (e.g., propylene glycol), azones (e.g., laurocapram), urea, fatty acids, surfactants, and terpenes.

According to one embodiment, the system may comprise further adjuvants, such as substances to reduce or prevent skin irritation.

According to one embodiment, the system comprises one or more vitamins.

In an advantageous embodiment, the combination of the alpha-hydroxy acid, the nitric oxide source and the further components described above show a good biocompatibility and reduce or prevent irritation of the epithelial layer to which the system is applied.

### The Final Composition

According to one embodiment, the system is for forming a final composition comprising the alpha-hydroxy acid and the nitric oxide source on the epithelial layer.

According to one embodiment, the final composition is a viscous solution, such as a gel, or a liquid solution. The final composition thus can be provided in any formulation, such as gel, cream, lotion, foam, or spray.

According to one embodiment, the alpha-hydroxy acid is present in the final composition at a concentration in the range of 0.05% to 20% (w/w), preferably 0.1% to 15% (w/w), more preferably 1% to 10% (w/w), most preferably 3% to 6% (w/w). In certain embodiments, the alpha-hydroxy acid is present in the final composition at a concentration of at least 2.5%, in particular at least 3%.

According to one embodiment, the concentration of the nitric oxide source in the final composition is in the range of 0.0001% to 1% (w/w), preferably 0.001% to 0.1% (w/w), more preferably 0.001% to 0.01% (w/w).

The pH value of the final composition may in particular be in the range of 1 to 7.4, especially in the range of 3.5 to 6.

### The Two-Component System

In certain embodiments, the system of the first aspect is a two-component system comprising a first composition, comprising an alpha-hydroxy acid, and a second composition, comprising a nitric oxide source.

According to one embodiment, the system comprises
(a) a first composition, wherein the first composition comprises the alpha-hydroxy acid; and
(b) a second composition, wherein the second composition comprises the nitric oxide source.

### The First Composition

According to one embodiment, the first composition of the two-component system is a viscous solution or a liquid solution, such as an aqueous solution. This is particularly advantageous in case the system is used in a formulation, such as gel, cream, lotion, foam, or spray.

According to one embodiment, the concentration of the alpha-hydroxy acid in the first composition is in the range of 1% to 50% (w/w), preferably 3% to 20% (w/w), more preferably 5% to 10% (w/w). In certain embodiments, the concentration of the alpha-hydroxy acid in the first composition is at least 5%, in particular at least 6%. According to these embodiments, the alpha-hydroxy acid preferably is glycolic acid.

According to one embodiment, the first composition may comprise further components. Such further components are described above in conjunction with the system and may be advantageously incorporated in the first composition of the two-component system.

According to one embodiment, the first composition further comprises one or more of the following components:
(a) ascorbic acid;
(b) at least one hyaluronic acid, wherein optionally the hyaluronic acid is provided by at least one low molecular weight hyaluronic acid and at least one high molecular weight hyaluronic acid;
(c) a composition comprising one or more vitamins, wherein preferably the composition is a liquid solution;
(d) polyacrylic acid; and/or
(e) water.

According to one embodiment, the first composition comprises all of the further components.

According to one embodiment, the at least one hyaluronic acid is provided by at least one low molecular weight hyaluronic acid. For example, the low molecular weight hyaluronic acid may have a molecular weight in the range of 20-200 kDa and/or the high molecular weight hyaluronic acid may have a molecular weight in the range of 500-2000 kDa.

According to one embodiment, the one or more vitamins is provided by a composition comprising vitamin A, vitamin C and vitamin E. Preferably said composition is a liquid solution.

According to one embodiment, the first composition is an aqueous solution, comprising
(i) the ascorbic acid at a concentration in the range of 0.1% to 1.0% (w/w), preferably 0.3% to 0.5% (w/w);
(ii) the low molecular weight hyaluronic acid at a concentration in the range of 0.1% to 2% (w/w), preferably 0.5% to 1.5% (w/w) and the high molecular weight hyaluronic acid at a concentration in the range of 0.001% to 1% (w/w), preferably 0.01% to 0.2% (w/w);
(iii) the liquid solution comprising vitamin A, vitamin C and vitamin E at a concentration in the range of 0.001% to 1% (w/w), preferably 0.01% to 0.2% (w/w); and
(iv) the polyacrylic acid at a concentration in the range of 1% to 10% (w/w), preferably, 2% to 5% (w/w).

### The Second Composition

According to one embodiment, the second composition of the two-component system is a viscous solution or a liquid solution, such as an aqueous solution. This is particularly advantageous in case the system is used in a formulation, such as gel, cream, lotion, foam, or spray.

According to one embodiment, the concentration of the nitric oxide source in the second composition is in the range of 0.0001% to 0.1% (w/w), 0.001% to 0.01% (w/w), preferably 0.002% to 0.005% (w/w), and wherein the nitric oxide source preferably is sodium nitrite (NaNO₂).

According to one embodiment, the second composition may comprise further components. Such further components are described above in conjunction with the system and may be advantageously incorporated in the second composition of the two-component system except the activator substance. In particular, the second composition may comprise a solvent and a thickening agent.

According to one embodiment, the second composition further comprises one or both of the following components:
(a) polyacrylic acid; and/or
(b) water.

According to one embodiment, the second composition comprises the polyacrylic acid at a concentration in the range of 0.1% to 10% (w/w), preferably 0.5% to 2.5% (w/w).

As is demonstrated by the examples, the combination of such a second composition and the first composition described above results in the generation and release of nitric oxide which is then transported and delivered by the alpha-hydroxy acid comprised in the first composition, such as glycolic acid, into and through the epidermis of the skin to deeper skin layers.

As disclosed herein, the first and the second compositions are combined to generate and release nitric oxide from the nitric oxide source which is then transported and delivered by the alpha-hydroxy acid through and into the different layers of the skin. This is for example achieved by topically applying the first composition and the second composition to the same area on an epithelial layer, such as the skin. Alternatively, the first and second compositions are mixed prior to topical application on the epithelial layer. In certain embodiments, the first composition is contacted with the second composition in a ratio of first composition to second composition that is selected from a range between 10:1 to 1:10, preferably 5:1 to 1:5, 4:1 to 1:4, 3:1 to 1:3, or 2:1 to 1:2, more preferably 1:1.

### Further Embodiments of the System According to the First Aspect

According to one embodiment, the system is a viscous solution or a liquid solution. This is particularly advantageous in case the system is used in a formulation, such as gel, cream, lotion, foam, or spray. According to this embodiment, the one-component system may comprise further additives to prevent nitric oxide generation, degradation, and/or release until the system is applied to the epithelial layer. For instance, nitric oxide may be generated upon contact to oxygen comprised in the atmosphere and/or upon contact to the skin.

According to one embodiment, the system comprises a first composition, comprising an alpha-hydroxy acid, and a second composition, comprising a nitric oxide source, wherein the first composition is a viscous solution, such as a gel, or a liquid solution, and wherein the second composition is a viscous solution, such as a gel, or a liquid solution. This embodiment is particularly preferred in case the system is applied directly to the epithelial layer, such as the skin and/or the mucosa. For instance, the first composition is provided by a first gel comprising the alpha-hydroxy acid and the second composition is provided by a second gel comprising the nitric oxide source. Preferred embodiments of the first composition and the second composition have been described elsewhere herein and it is referred to the respective disclosure. The two gels may be applied to the same area on the epithelial layer, such as the skin or the mucosa, and are thereby contacted, i.e. mixed. Upon contact nitric oxide is generated from the nitric oxide source comprised in the second gel and caught by the alpha-hydroxy acid comprised in the first gel. The alpha-hydroxy acid carrying the nitric oxide transports the nitric oxide through the epithelial layer and delivers it to deeper tissue layers, e.g. the dermis and/or hypodermis.

According to one embodiment, the system comprises
(a) a first composition, wherein the first composition comprises at least one alpha-hydroxy acid as defined in conjunction with the alpha-hydroxy acid according to the first aspect above,
   wherein the first composition is a viscous solution or a liquid solution, preferably wherein the first composition is a gel, and
   wherein preferably the at least one alpha-hydroxy acid in the first composition is glycolic acid, wherein the concentration of the glycolic acid in the first composition is in the range of 1% to 20% (w/w), 5% to 15% (w/w), preferably 5% to 10%; and
(b) a second composition, wherein the second composition comprises at least one nitric oxide source as defined in conjunction with the nitric oxide source according to the first aspect above, wherein the first composition is a viscous solution or a liquid solution, preferably wherein the second composition is a gel, and
   wherein preferably the at least one nitric oxide source in the second composition is sodium nitrite (NaNO₂), wherein the concentration of sodium nitrite (NaNO₂) in the second composition is in the range of 0.0001% to 0.1% (w/w), 0.001% to 0.01% (w/w), preferably 0.002% to 0.005% (w/w).

The first and second compositions of the two-component system according to the first aspect are maintained separately until use. As is demonstrated by the examples, such a two-component system is suitable to generate and release nitric oxide upon contact of the first composition and the second composition, in particular under physiological conditions at 37° C. Furthermore, the alpha-hydroxy acid comprised in the two component system transports and delivers the generated nitric oxide through and into the epidermis of the skin to deeper skin layer, which is demonstrated in the examples by the improvement of the skin condition acne vulgaris.

The system according to the first aspect may be provided in any suitable container. The first composition and the second composition of the two-component system may be provided in separate containers or separate compartments of the same container. For instance, the system may be stored in a syringe, tube or flask. Accordingly, in case the system is provided as a two-component system, the first and second composition may each be provided in separate syringes, tubes or flasks, or in one syringe, tube or flask having two compartments. This may be especially advantageous in case the first and the second compositions are provided as viscous or liquid solutions.

In certain embodiments, the system according to the first aspect is provided in or as part of a dressing. In case the two-component system is provided in a dressing, the dressing may have two compartments. The barrier between the two compartments of the dressing may be destroyed when the dressing is prepared for application, e.g. when the protecting sheet is removed such that the first and the second composition can be mixed.

The container may be designed according to the type of formulation in which the system is provided, i.e. the system may be comprised in any formulation or preparation for topical administration e.g., gel, cream, lotion, foam, or spray. The formulation may also be provided as powder or lyophilisate which may be reconstituted in a solvent prior to topical application.

### Medical Applications

According to the second aspect, a system according to the first aspect is provided for use in medicine. In embodiments, medicine comprises human medicine and veterinary medicine. As explained above, nitric oxide is involved in many physiological and pathophysiological processes, including vasodilation, platelet and leukocyte aggregation and adhesion, cell proliferation, endothelial layer permeability, scavenging of superoxide radicals, antimicrobial effects, immunomodulatory effects, and wound repair. The system according to the first aspect is therefore suitable to regulate such processes by the provision of nitric oxide and the transport and delivery of the nitric oxide to the target sites.

According to a third aspect, a system according to the first aspect is provided for use in the treatment of infections, inflammations, injuries, skin disorders and/or blood vessel disorders, such as chronic inflammatory skin diseases, acne, eczema, neurodermatitis, psoriasis, scars, wrinkles, abrasions, burns, trauma, hematoma, androgenetic alopecia (male-pattern hair loss and female-pattern hair loss), blepharitis, Raynaud syndrome, bacterial skin & soft-tissue infections, leg ulcer causes by peripheral vascular disease, chronic venous insufficiency and/or ischemic vasculitis. The subjects to be treated may be humans or animals, especially humans. As is demonstrated by the examples, the system of the present invention is particularly suitable for the use in the treatment of a human skin disorder, such as acne. Accordingly, the system can be used in the treatment of infections, inflammations, injuries, skin disorders and/or blood vessel disorders of any mammal, such as dogs, cats, horses, cows, sheeps, pigs, etc.

According to one embodiment, the system is administered topically on an epithelial layer, such as skin and/or mucosa. According to this aspect, the system according to the first aspect can be topically administered or applied to an epithelial layer located at the site of the physiological or pathophysiological process to be regulated as described above. As demonstrated by the examples, the system according to the first aspect can be used for topical administration on the human skin. The system may be directly administered to the target area on the epithelial layer. According to an advantageous embodiment, the system is a viscous solution or a liquid solution as described elsewhere herein. In case the two-component system is used, the first and second composition can be administered in any order, i.e. either the first composition is topically administered first on an area of an epithelial layer and then the second composition is administered to the same area or the second composition is administered first and then the first composition. It is advantageously to distribute the two compositions homogeneously on the area of the epithelial layer to ensure optimal mixture of both compositions and thus optimal nitric oxide generation. Consequently, the amount of nitric oxide transported and delivered by the alpha-hydroxy acid is maximized.

According to one embodiment, the system induces vasodilation, increases blood flow, and/or increases angiogenesis. As explained above, nitric oxide is a potent vasodilator and plays an important role in regulating vascular local resistance which is often impaired during infections, inflammations, injuries, and skin and blood vessel disorders.

According to one embodiment, the system is first applied on a skin and/or mucosa dressing, such as patches, plasters, bandings, pads, compresses, and folios, and then topically administered to an epithelial layer, such as skin and/or mucosa. This embodiment is advantageous in case a dressing is necessary to cover the area of the target site. This may be the case when bleeding occurs and absorbance of blood, pus and/or lymph is required or when the target area needs to be protected, e.g. from pathogens in case of injury.

According to one embodiment, upon topical administration of the system on the epithelial layer nitric oxide is generated and/or released. Suitable conditions leading to the generation and/or release of nitric oxide from the nitric oxide source of the system, such as an acidic pH, are described elsewhere herein and it is referred to the respective disclosure. The generated nitric oxide is subsequently caught by the alpha-hydroxy acid of the system of the present invention and transported into and/or through the epithelial layer and delivered to the target site in the deeper tissue layers.

In certain embodiments, the use in medicine comprises the steps of
(i) optionally, if a two-component system is used, mixing the first composition and the second composition of the two-component system,
(ii) applying the system to the epithelial layer, and
(iii) activating the nitric acid source to generate and/or release nitric oxide.

The steps of the method may be performed in any sequence. The three steps are in particular initiated shortly after each other or even simultaneous, especially within 30 minutes, preferably within 5 minutes, more preferably within 1 minute. Especially, if a two-component system is used, the nitric acid source is activated to generate and/or release nitric oxide by mixing the first and the second composition.

### Dressings

According to a fourth aspect, a dressing is provided comprising the system according to the first aspect. As explained above, a dressing may be used to absorb body fluids, such as blood, or to protect the target area on the epithelial layer.

According to one embodiment, the dressing is selected from the group consisting of patches, plasters, bandings, pads, compresses, and folios.

In certain embodiments, the two-component system is described herein is used in the dressing. In these embodiments, the two components of the system, the first and second composition, are present in separate compartments of the dressing and only mixed upon application of the dressing to the skin and/or mucosa of a subject, or upon preparing the dressing for said application.

Methods and uses for enhancing the transport and/or delivery of nitric oxide

According to a fifth aspect, the present invention provides a method for enhancing the transport and/or delivery of nitric oxide into and/or through an epithelial layer, such as skin and/or mucosa, the method comprising the steps of:
(i) mixing a nitric oxide source with an alpha-hydroxy acid,
(ii) applying the nitric acid source to the epithelial layer,
(iii) activating the nitric acid source to generate and/or release nitric oxide.

The transport and/or delivery of nitric oxide into and/or through the epithelial layer is enhanced by the presence of the alpha-hydroxy acid compared to the same method not comprising step (i).

The steps of the method may be performed in any sequence. Performing the steps in the sequence of (i), (ii) and (iii) is preferred. If another sequence is used, the three steps are in particular initiated shortly after each other or even simultaneous, especially within 30 minutes, preferably within 5 minutes, more preferably within 1 minute.

According to a sixth aspect, the use of an alpha-hydroxy acid for enhancing the transport and/or delivery of nitric oxide into and/or through an epithelial layer, such as skin and/or mucosa, is provided.

The features, properties and embodiments of the system described herein likewise apply to the method for enhancing the transporting and/or delivering of nitric oxide and to the use of the alpha-hydroxy acid.

According to one embodiment, the alpha-hydroxy acid is selected from the group consisting of glycolic acid, lactic acid, citric acid, isocitric acid, citric acid monohydrate, mandelic acid, tartaric acid, malic acid, tartronic acid, hydroxycaprylic acid, hydroxycapric acid and mixtures thereof. Preferred embodiments of the alpha-hydroxy acid have been described elsewhere herein and it is referred to the respective disclosure.

As used in the subject specification, items and claims, the singular forms “a”, “an” and “the” include plural aspects unless the context clearly dictates otherwise. The terms “include,” “have,” “comprise” and their variants are used synonymously and are to be construed as non-limiting. Further components and steps may be present. Throughout the specification, where compositions are described as comprising components or materials, it is additionally contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

### SPECIFIC EMBODIMENTS

In the following, specific embodiments of the present invention are described.

Embodiment 1. A system for transporting and/or delivering of nitric oxide into and/or through an epithelial layer, such as skin and/or mucosa, comprising an alpha-hydroxy acid and a nitric oxide source.

Embodiment 2. The system according to embodiment 1, wherein the epithelial layer is the epidermis of the skin.

Embodiment 3. The method according to embodiment 2, wherein the nitric oxide is transported and/or delivered into the dermis and/or hypodermis.

Embodiment 4. The system according to any one of embodiments 1 to 3, wherein the alpha-hydroxy acid is selected from the group consisting of glycolic acid, lactic acid, citric acid, isocitric acid, citric acid monohydrate, mandelic acid, tartaric acid, malic acid, tartronic acid, hydroxycaprylic acid, hydroxycapric acid and mixtures thereof.

Embodiment 5. The system according to embodiment 4, wherein the alpha-hydroxy acid is glycolic acid.

Embodiment 6. The system according to any one of embodiments 1 to 5, wherein the nitric oxide source is selected from the group consisting of authentic nitric oxide, nitric oxide solution, organic and inorganic nitrates, such as mono-nitrate, di-nitrate and tri-nitrate, acidified nitrite, nitrogen oxides, and mixtures thereof.

Embodiment 7. The system according to embodiment 6, wherein the nitric oxide source is an acidified inorganic nitrite.

Embodiment 8. The system according to embodiment 6 or 7, wherein the nitric oxide source is sodium nitrite (NaNO₂).

Embodiment 9. The system according to any one of embodiments 1 to 8, wherein the system further comprises an activator substance which initiates release of nitric oxide from the nitric oxide source.

Embodiment 10. The system according to any one of embodiments 1 to 9, wherein the system further comprises a solvent and optionally a thickening agent, such as a gelling agent.

Embodiment 11. The system according to any one of embodiments 1 to 10, wherein the system is for forming a final composition comprising the alpha-hydroxy acid and the nitric oxide source on the epithelial layer.

Embodiment 12. The system according to embodiment 11, wherein the final composition is a viscous solution, such as a gel, or a liquid solution.

Embodiment 13. The system according to embodiment 11 or 12, wherein the alpha-hydroxy acid is present in the final composition at a concentration in the range of 0.05% to 20% (w/w), preferably 0.1% to 15% (w/w), more preferably 1% to 10% (w/w), most preferably 3% to 6% (w/w).

Embodiment 14. The system according to embodiment 11 or 12, wherein the alpha-hydroxy acid is present in the final composition at a concentration of at least 2.5%.

Embodiment 15. The system according to embodiment 11 or 12, wherein the alpha-hydroxy acid is present in the final composition at a concentration of at least 3%.

Embodiment 16. The system according to any one of embodiments 11 to 15, wherein the concentration of the nitric oxide source in the final composition is in the range of 0.0001% to 1% (w/w), preferably 0.001% to 0.1% (w/w), more preferably 0.001% to 0.01% (w/w).

Embodiment 17. The system according to any one of embodiments 1 to 16, wherein the system comprises
(a) a first composition, wherein the first composition comprises the alpha-hydroxy acid; and
(b) a second composition, wherein the second composition comprises the nitric oxide.

Embodiment 18. The system according to embodiment 17, wherein the concentration of the alpha-hydroxy acid in the first composition is in the range of 1% to 20% (w/w), preferably 5% to 15% (w/w), more preferably 5% to 10% (w/w).

Embodiment 19. The system according to embodiment 17, wherein the concentration of the alpha-hydroxy acid in the first composition is at least 5%.

Embodiment 20. The system according to embodiment 17, wherein the concentration of the alpha-hydroxy acid in the first composition is at least 6%.

Embodiment 21. The system according to any one of embodiments 17 to 20, wherein the alpha-hydroxy acid is glycolic acid.

Embodiment 22. The system according to any one of embodiments 17 to 21, wherein the concentration of the nitric oxide source in the second composition is in the range of 0.0001% to 0.1% (w/w), 0.001% to 0.01% (w/w), preferably 0.002% to 0.005% (w/w).

Embodiment 23. The system according to any one of embodiments 17 to 22, wherein the nitric oxide source is sodium nitrite (NaNO₂).

Embodiment 24. The system according to any one of embodiments 17 to 23, wherein the first composition is a viscous solution, such as a gel, or a liquid solution, and wherein the second composition is a viscous solution, such as a gel, or a liquid solution.

Embodiment 25. The system according to any one of embodiments 17 to 24, wherein the first composition and the second composition in the system have a volume ratio in the range of 10:1 to 1:10, preferably 5:1 to 1:5, 4:1 to 1:4, 3:1 to 1:3, or 2:1 to 1:2, more preferably 1:1.

Embodiment 26. The system according to any one of embodiments 1 to 25 for use in medicine.

Embodiment 27. The system according to any one of embodiments 1 to 25 for use in the treatment of infections, inflammations, injuries, skin disorders and/or blood vessel disorders, such as chronic inflammatory skin diseases, acne, eczema, neurodermatitis, psoriasis, scars, wrinkles, abrasions, burns, trauma, hematoma, androgenetic alopecia (male-pattern hair loss and female-pattern hair loss), blepharitis, Raynaud syndrome, bacterial skin & soft-tissue infections, leg ulcer causes by peripheral vascular disease, chronic venous insufficiency and/or ischemic vasculitis.

Embodiment 28. The system for use in medicine according to embodiment 26 or 27, wherein the system is administered topically on an epithelial layer, such as skin and/or mucosa.

Embodiment 29. The system for use in medicine according to any one of embodiments 26 to 28, wherein the system induces vasodilation, increases blood flow, and/or increases angiogenesis.

Embodiment 30. The system for use in medicine according to any one of embodiments 26 to 29, wherein the system is first applied on a skin and/or mucosa dressing, such as patches, plasters, bandings, pads, compresses, and folios, and then topically administered to an epithelial layer, such as skin and/or mucosa;

Embodiment 31. The system for use in medicine according to any one of embodiments 26 to 30, wherein upon topical administration of the system on the epithelial layer nitric oxide is generated and/or released.

Embodiment 32. The system for use in medicine according to any one of embodiments 26 to 31, wherein the use in medicine comprises the steps of
(i) optionally, if a two-component system is used, mixing the first composition and the second composition of the two-component system,
(ii) applying the system to the epithelial layer, and
(iii) activating the nitric acid source to generate and/or release nitric oxide.

Embodiment 33. A dressing comprising the system according to any one of embodiments 1 to 25.

Embodiment 34. The dressing according to embodiment 33, wherein the dressing is selected from the group consisting of patches, plasters, bandings, pads, compresses, and folios.

Embodiment 35. A method for enhancing the transport and/or delivery of nitric oxide into and/or through an epithelial layer, such as skin and/or mucosa, the method comprising the steps of:
(i) mixing a nitric oxide source with an alpha-hydroxy acid,
(ii) applying the nitric acid source to the epithelial layer,
(iii) activating the nitric acid source to generate and/or release nitric oxide.

Embodiment 36. The method according to embodiment 35, having one or more of the features as defined in any one of embodiments 2 to 25.

Embodiment 37. Use of an alpha-hydroxy acid for enhancing the transporting and/or delivering of nitric oxide into and/or through an epithelial layer, such as skin and/or mucosa.

Embodiment 38. The use according to embodiment 37, wherein the alpha-hydroxy acid is selected from the group consisting of glycolic acid, lactic acid, citric acid, isocitric acid, citric acid monohydrate, mandelic acid, tartaric acid, malic acid, tartronic acid, hydroxycaprylic acid, hydroxycapric acid and mixtures thereof.

Embodiment 39. The use according to embodiment 37 or 38, having one or more of the features as defined in any one of embodiments 2 to 25.

### EXAMPLES

It should be understood that the following examples are for illustrative purpose only and are not to be construed as limiting this invention in any manner.

Exemplarily we describe herein the use of the transport delivery system of the present invention within a two-component system using acidified inorganic nitrite, which has been described for the generation of NO in topical applications.

### Example 1: Preparation of Acidified Nitrite Gel for Topical Release of NO

The following, non-limiting example illustrates the use of the transport delivery system in a two-component system for topical application. It is a two-component gel (Gel A and B) with glycolic acid as the relevant AHA, reductant, granulation agent and a penetration enhancer (Gel A) and a nitric oxide source (Gel B). After merging of Gel A and B, a controlled in situ NO generation is initiated and the included AHA transports the NO through the skin layers to the target site.

The two components Gel A and Gel B (A containing AHA and B containing nitrite) are mixed in individually and maintained separately until use.

To prepare the acidic gel, 50 ml deionized water was heated to 40° C. 0.21 g of ascorbic acid and 4.5 g of glycolic acid were added and the solution stirred for 2 min until ascorbic acid was completely dissolved. 0.5 g hyaluronic acid (50 kDa) and 0.05 g hyaluronic acid (1500 kDa) were added and stirred until complete dissolution. Next, 2 g of polyacrylic acid (PAA) were added and mixed until PAA was completely gelled. Finally, 0.05 g of a vitamin A/C/E fluid were added (since vitamin A and vitamin E are insoluble in water, a soluble active complex consisting of aqua, pentylene glycole, lecithine, caprylic/capric triglyceride, tocopherol acetate, retinyl palmitate, sodium phytate, sodium ascorbyl phosphate, sodium hydroxide was used).

To prepare the nitrite gel, 0.055 g sodium nitrite was solved in 50 ml deionized water and stirred until complete dissolution. Next 0.7 g of polyacrylic acid (PAA) was added and the mixture was stirred until PAA was completely gelled.

After preparation Gel A and Gel B were filled into syringes of 10 ml volume and carefully closed. Application to the skin is carried out with the same amount of both gels leading to a 1:1 ratio of Gel A and Gel B in the applied mixture.

TABLE 1
Components of composition A

| | | n | mass |
|---|---|---|---|
| Ingredients | m (g) | (mmol) | fraction (%) |
| water | 50 | — | 87.2 |
| ascorbic acid | 0.21 | 1.192 | 0.4 |
| glycolic acid | 4.5 | 59.172 | 7.8 |
| hyaluronic acid (50 kDa) | 0.5 | 0.010 | 0.9 |
| hyaluronic acid (1500 kDa) | 0.05 | 0.00003 | 0.1 |
| Vitamin A/C/E fluid | 0.05 | — | 0.1 |
| polyacrylic acid (PAA) | 2 | 27.755 | 3.5 |

TABLE 2
Components of composition B

| | | n | mass |
|---|---|---|---|
| Ingredients | m (g) | (mmol) | fraction (%) |
| water | 50 | — | 98.6 |
| sodium nitrite (NaNO2) | 0.055 | 0.8 | 0.0034 |
| polyacrylic acid (PAA) | 0.7 | 9.7 | 1.381 |

### Example 2: NO-Generation of Activated Gel-Formation

In-vitro NO production capabilities of the two-component gel were investigated using a nitric oxide chemiluminescent detector (CLD). 1 ml of both components were added to a gas-tight reaction chamber. The reaction chamber contains an inlet port for the inert carrier gas helium (99.996% He; flow rate: 124 ml/min., pressure controlled) and an outlet port leading to the analyzer. In order to create a physiological environment, the reaction chamber was heated to 37° C. and to ensure optimum mixing of both components, a magnetic stirrer was used.

NO evolving the reaction as a result was determined using an ozone-based chemiluminescence assay. NO was actively purged with an inert helium stream in-line with a NO chemiluminescence analyzer (ANALYZER CLD 88 sp; Eco Physics) and quantified in the gas phase after reaction with ozone. Provided O₃ is present in excess and reaction conditions are kept constant, the intensity of light emitted is directly proportional to NO concentration.

### Results

The mixture of both components resulted in stable generation of NO, exceeding the upper limit of detection of 23.847 ppb NO, resulting in a plateau phase of about 15 minutes after activation time of approximately 60 seconds (FIG. 2). NO generation was, although decreasing, still detectable 30 min after initial application. When applied on the skin, measurements on 7 cm² intact skin showed comparable generation of NO.

### Example 3: Application of the Transport Delivery System in NO-Generating Gel Against Acne Vulgaris

Facial acne vulgaris is the most common skin disease in adolescents and adults and a primary inflammatory disorder of the pilosebaceous unit of multifactorial etiology. Within a physiological range, NO possesses a concentration-dependent antimicrobial and immunomodulatory bimodal activity. Using higher concentrations in topical application, NO demonstrates anti-inflammatory and antimicrobial properties. The use of the transport delivery system allows for better and deeper transportation of NO into the skin layers and improved acne disease in representative patient examples.

A total of 10 consecutive patients suffering from mild to moderate facial acne vulgaris were enrolled in the studies. The mean age was 34.4 years. The transport delivery system was applied in a gel-formulation in a thin layer twice daily on the skin for 8 consecutive weeks. Clinical scores were evaluated by trained examiners based on high-resolution images.

### Results

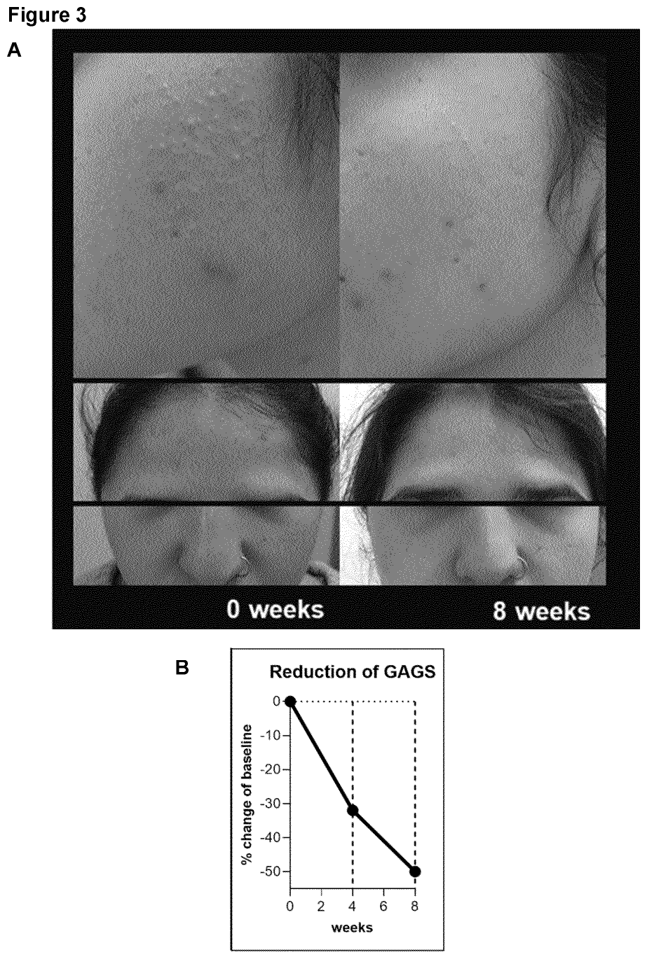

Eight weeks of application demonstrated clinical improvement of acne vulgaris. Application reduced the amount of closed and open comedones and inflammatory pustulae in moderate acne vulgaris, resulting in a purer skin texture (FIG. 3A).

The Global Acne Grading System (GAGS) score was reduced by about 50% after eight weeks of treatment (FIG. 3B).

1. A system for transporting and/or delivering of nitric oxide into and/or through an epithelial layer comprising an alpha-hydroxy acid and a nitric oxide source.

2. The system according to claim 1, wherein the epithelial layer is the epidermis of the skin and wherein the nitric oxide is transported and/or delivered into the dermis and/or hypodermis.

3. The system according to claim 1, wherein the alpha-hydroxy acid is selected from the group consisting of glycolic acid, lactic acid, citric acid, isocitric acid, citric acid monohydrate, mandelic acid, tartaric acid, malic acid, tartronic acid, hydroxycaprylic acid, hydroxycapric acid and mixtures thereof.

4. The system according to claim 1, wherein the nitric oxide source is selected from the group consisting of authentic nitric oxide, nitric oxide solution, organic and inorganic nitrates, such as mono-nitrate, di-nitrate and tri-nitrate, acidified nitrite, nitrogen oxides, and mixtures thereof.

5. The system according to claim 1, wherein the alpha-hydroxy acid is glycolic acid and the nitric oxide source is sodium nitrite (NaNO₂).

6. The system according to claim 1, wherein the system further comprises an activator substance which initiates release of nitric oxide from the nitric oxide source and a solvent.

7. The system according to claim 1, wherein the system is for forming a final composition comprising the alpha-hydroxy acid and the nitric oxide source on the epithelial layer.

8. The system according to claim 7, wherein the alpha-hydroxy acid is present in the final composition at a concentration in the range of 0.05% to 20% (w/w), and wherein the concentration of the nitric oxide source in the final composition is in the range of 0.0001% to 1% (w/w).

9. The system according to claim 1,
wherein the system comprises
(a) a first composition, wherein the first composition comprises the alpha-hydroxy acid; and
(b) a second composition, wherein the second composition comprises the nitric oxide.

10. A method of treating an infection, inflammation, injury, skin disorder, and/or blood vessel disorder in a patient, comprising administering to the patient the system according to claim 1.

11. The method according to claim 10 wherein the infection, inflammation, injury, skin disorder, and/or blood vessel disorder is selected from the group consisting of chronic inflammatory skin diseases, acne, eczema, neurodermatitis, psoriasis, scars, wrinkles, abrasions, burns, trauma, hematoma, androgenetic alopecia (male-pattern hair loss and female-pattern hair loss), blepharitis, Raynaud syndrome, bacterial skin and soft-tissue infections, leg ulcer causes by peripheral vascular disease, chronic venous insufficiency and/or ischemic vasculitis.

12. The method according to claim 10, wherein the system is administered topically on an epithelial layer.

13. A dressing comprising the system according to claim 1.

14. The dressing according to claim 13, wherein the dressing is selected from the group consisting of patches, plasters, bandings, pads, compresses, and folios.

15. A method for enhancing the transport and/or delivery of nitric oxide into and/or through an epithelial layer, the method comprising the steps of:
(i) mixing a nitric oxide source with an alpha-hydroxy acid,
(ii) applying the nitric acid source to the epithelial layer, and
(iii) activating the nitric acid source to generate and/or release nitric oxide.

16. (canceled)

17. The system according to claim 6, wherein the system further comprises a thickening agent.

18. The system according to claim 17, wherein the thickening agent is a gelling agent.

19. The system of claim 1, wherein the epithelial layer is skin and/or mucosa.

20. The system of claim 12, wherein the epithelial layer is skin and/or mucosa.

21. The method of claim 15, wherein the epithelial layer is skin and/or mucosa.
